(19) United States
(12) Patent Application Publication
(10) Pub. No.: US 2021/0361786 A1
(43) Pub. Date: Nov. 25, 2021

(54) MATERIAL WITH SUPERCAPACITANCE MODIFIED SURFACE AND PREPARATION METHOD AND APPLICATION THEREOF

(71) Applicant: China Morefound Technology Ltd., Shenzhen, Shenzhen, Guangdong (CN)

(72) Inventors: Guomin WANG, Shenzhen, Guangdong (CN); Huaiyu WANG, Shenzhen, Guangdong (CN); Liangsheng HU, Shenzhen, Guangdong (CN); Kimho Paul CHU, Shenzhen, Guangdong (CN)

(21) Appl. No.: 16/606,549

(22) PCT Filed: Mar. 1, 2019
(86) PCT No.: PCT/CN2019/076721

§ 371 Date: Oct. 18, 2019

(30) Foreign Application Priority Data
May 14, 2018 (CN) 201810454974.5

(51) Int. Cl. A61L 2/03; A61L 2/26; C23F 17/00; H01G 11/46; H01G 11/86

(52) U.S. Cl. CPC A61L 2/03; A61L 2/26; C25D 11/26; H01G 11/46; H01G 11/86; C23F 17/00

(57) ABSTRACT

Disclosed are a material with supercapacitance modified surface and a preparation method and application thereof. Specifically, the present disclosure introduces a material having a controllably supercapacitive surface. The surface is chargeable, the full-charged modified surface can interact with bacteria disturbing the electron transfer of respiratory chain of bacteria and inhibiting the growth and reproduction of bacteria in a short-term. The antibacterial rate can be improved by cyclically charging-discharging without losing capacitance, and prevent formation of biofilm of bacteria. The antibacterial system can quantitatively control the antibacterial process without affecting the biocompatibility of the material, and has the advantages of environmental protection and controllability.

### TECHNICAL FIELD

The present disclosure relates to a surface modified material, in particularly to a material with supercapacitance modified surface and a preparation method and application thereof.

### BACKGROUND

With the development of clinical medicine and materials science, new medical metal implant materials are widely used in clinical practice. Especially in the field of orthopedics, medical metal implant material such as blade plates, intramedullary nails, screw-rod system, artificial periprosthetic joint and the like which are used as intraosseous metal implants and hard tissue repairing materials have been widely used in clinical applications. However, for living organisms, medical metal implant materials are still foreign objects, and there are big differences between them and the internal environment in terms of physical and chemical properties.

Medical alloy implant materials are particularly prone to the following three problems during use: \textcircled{1} the potential cell biological toxicity and tissue and organ damage caused by precipitation of trace elements from alloys; \textcircled{2} the local inflammation caused by possible abrasion between the materials of periprosthetic joint with the increase of service life; \textcircled{3}the risk of infection associated with the implant materials and looseness of transplant caused by formation of bacterial biofilm, etc. Implant-related infections are especially tricky for surgeons, which may cause disastrous consequence if it occurs.

Titanium alloys are widely used in biomedical field as implant material due to their excellent biocompatibility, corrosion resistance and appropriate mechanical properties. However, the existing titanium alloy-based biomedical materials do not have antibacterial properties. The bacteria in human body will grow and form extracellular polymer matrix biofilm with specific structure and stronger resistance, which may lead to the failure of transplantation surgery and cause serious postoperative infection, the patients may have to suffer the illness or even death. Therefore, the designed surface of titanium alloy with antibacterial properties can effectively solve the above problems plaguing doctors and patients. The original design of antibacterial material is grafting antibiotics or antibacterial peptides on the surface of material to achieve effective antibacterial effect. Moreover, material surface modified with nano-gold, silver and graphene also have antimicrobial effect. Further researches show that electron transfer between the platform and the bacteria should play a key role in this type of antibacterial process. some studies present that the surface of charged material is also antibacterial due to electron transfer. All methods above promote the development of antibacterial materials step by step. (Wang, G. et al. Extracellular electron transfer from aerobic bacteria to Au-loaded TiO₂ semiconductor without light: a new bacteria-killing mechanism other than localized surface plasmon resonance or microbial fuel cells. ACS Appl. Mater Interfaces 8, 24509-24516 (2016). Chernousova, S., Epple, M. Silver as antibacterial agent: ion, nanoparticle, and metal. Angew. Chem. Int. Ed. 52, 1636-1653 (2013)).

By the surface design of titanium alloy material, effective antibacterial action can be achieved, and thus the success rate of its biomedical applications can be improved. However, the current design of the antibacterial materials has the following defects, for example, the grafting of antibiotics and antibacterial peptides on the surface of the materials can cause serious bacterial resistance, and mutations in resistant strains can aggravate clinical infections. At the same time, various peptide materials are susceptible to immune reaction with the body, and thus the risk of implantation surgery failure can increase. Although the above-mentioned antibacterial surface design that relies on nano-materials and the method of modifying charge to the surface of the material can effectively avoid mutation of the resistant strain and reduce the immune reaction by directly interfering with the electron transfer of the bacterial respiratory chain to inhibit bacterial growth. The present antimicrobial surface designs can't quantitatively control electron transfer, and the antimicrobial system can't be cyclically used. In addition, the introduction of silver nanoparticles and quaternary ammonium salts will also reduce the biocompatibility of the material and resulting to its slow applicability in vivo.

### SUMMARY

The object of the present disclosure is to design an antibacterial system based on materials with supercapacitive characteristics, which uses those materials to modify the surface of a metal platform, and the post-charging platform will transfer electron with bacteria without any intervention to achieve the quantitative control of electron transfer between platform and bacteria, thus acquiring stronger antibacterial effect. This clean and environmentally friendly antibacterial system overcomes the defects of potential risk of existing antibacterial materials and the inability to quantitatively control.

The technical solution adopted by the present disclosure is:

The present disclosure includes three aspects. Firstly, the design and manufacture of supercapacitance modified surface, and charging it in circuit to full power, then disconnecting the power and interacting with bacteria, to disturb electron transfer of respiratory chain of microorganism in a short-term and inhibit growth and reproduction of bacteria.

Specifically, the first aspect of the present disclosure relates to a material with supercapacitance modified surface comprising a material body and a supercapacitance layer on its surface; wherein, the material body is selected from a metal material or other conductor; wherein, the metal material is preferably titanium or an alloy thereof, aluminum or an alloy thereof, stainless steel, nickel or an alloy thereof, manganese or an alloy thereof, tungsten or an alloy thereof, and zinc or an alloy thereof; the other conductors include, but are not limited to, conductive polymers, and examples of the conductive polymers include polypyrrole, polyacetylene, polythiophene, and polyaniline, and the like.

The supercapacitance layer refers to a functional layer having a surface capacitance greater than 10 mF·cm⁻²; the surface capacitance of the supercapacitance layer is greater than 50 mF·cm⁻², preferably greater than 100 mF·cm⁻².

Further, the metal material is preferably a titanium alloy, an aluminum alloy, a stainless steel, a nickel alloy, and a zinc alloy; and the supercapacitance layer is preferably a titanium dioxide nanotube array layer, a zinc oxide nanorod layer, or a reduced graphene oxide.

As for the supercapacitance layer according to the present disclosure, the diameter of titanium dioxide nanotubes or zinc oxide nanorods is from 10 nm to 1000 nm, preferably 20 to 800 nm, most preferably 50 to 500 nm; the nanotubes or the nanorods have a length from 500 nm to 10 μm; preferably 800 nm to 5 μm; and most preferably 1 to 3 μm.

Most preferably, the titanium dioxide nanotube array layer further includes carbon deposition; and the zinc oxide nanorod layer is doped with silver, gold, copper or platinum nanoparticles.

Another aspect of the present disclosure relates to a preparation method of a supercapacitance modified surface of material, which specifically includes anodizing a surface of a metal material.

According to the preparation method of the present disclosure, it is preferred to polish and grind, and clean the metal material prior to the anodization.

According to the preparation method of the present disclosure, said clean is ultrasonic cleaning with acetone, alcohol, and deionized water in sequence.

According to the preparation method of the present disclosure, the electrolyte used for anodizing is a mixed liquor of an ammonium salt, a lower alcohol, water, and a polyol.

According to the preparation method of the present disclosure, the mass-to-volume ratio of the ammonium salt, the lower alcohol, the water, and the polyol in the electrolyte is (1-10%): (1-10%): (1-10%): (70-95%).

According to the preparation method of the present disclosure, the ammonium salt is selected from the group consisting of an ammonium halide, preferably ammonium fluoride, ammonium chloride, and ammonium bromide.

According to the preparation method of the present disclosure, the lower alcohol is selected from one or more of methanol, ethanol, n-propanol, isopropanol, n-butanol, isobutanol, and tert-butanol.

According to the preparation method of the present disclosure, the water used is deionized water.

According to the preparation method of the present disclosure, the polyol is selected from the group consisting of ethylene glycol or glycerol.

According to the preparation method of the present disclosure, the voltage of the anodization is 10 to 100 V, preferably 15 to 80 V, most preferably 30 to 60 V.

According to the preparation method of the present disclosure, the reaction time of the anodization is 20 to 1000 min; preferably 30 to 800 min; and most preferably 40 to 500 min.

According to the preparation method of the present disclosure, it further includes placing the nanotubes array obtained after anodization into a vacuum tube furnace, vacuum annealing to achieve carbon deposition which enhances capacitive properties of nanotubes array. The carbon source used in carbon deposition is an organic substance in the anodizing process, and the anodized sample is annealed in vacuum at a high temperature to carbon deposition.

The temperature of vacuum annealing is preferably 500-800° C., the annealing time is 1-5 h, and the heating rate is 1-20° C. min⁻¹.

Another aspect according to the present disclosure relates to a method of preparing metal material with supercapacitance modified surface, specifically, growing zinc oxide nanorods on the surface of metal material via a hydrothermal method and modifying the doped silver, gold, copper or platinum nanoparticles via magnetron sputtering, the specific steps are as follows:

preparation of zinc oxide seed crystal: dissolving zinc acetate and a strong base in a lower alcohol, quickly spin coating on the surface of a metal material to obtain a wet film, heating to volatilize solvent and pyrolyzing to obtain a crystal seed layer on metal material;

Growth of zinc oxide nanorods: placing the metal material with crystal seed into a reactor, adding a mixed aqueous solution of a zinc salt and a base, sealing the reactor and heating; then, sputtering silver, gold, copper or platinum nanoparticles via magnetron sputtering; the base is preferably hexamethylenetetramine, sodium hydroxide, potassium hydroxide, calcium hydroxide, and aqueous ammonia or a combination thereof.

The lower alcohol is selected one or more from methanol, ethanol, n-propanol, isopropanol, n-butanol, isobutanol, and tert-butanol; preferably methanol, ethanol or a combination thereof.

The zinc salt is selected from the group consisting of zinc nitrate, zinc sulfate, zinc acetate, zinc phosphate, or a combination thereof.

Another aspect according to the present disclosure relates to a preparation method of a supercapacitance modified surface on metal material, specifically, selecting a metal material as a working electrode to electrodeposition; mixing graphene oxide with aqueous alcohol solution as an electrodeposition solution, and connecting a reference electrode and a counter electrode, electrodeposition by direct current to obtain a graphene oxide layer; putting the graphene oxide layers into hydrazine solution to hydro-thermal treatment to obtain a reduced graphene oxide-metal composite.

Another aspect according to the present disclosure relates to a bactericidal method, characterized by using the material with supercapacitance modified surface described above.

The sterilization method according the present disclosure is following specific steps, charging the material with a direct current or an alternating current circuit and contacting the post-charging material with the bacterial solution.

The electric charge charged is preferably positive.

According to the sterilization method of the present disclosure, the peak of voltage of direct current or alternating current circuit is 2-40 V, and the frequency is from 1 Hz to 1 MHz.

According to the sterilization method of the present disclosure, the voltage set range of the circuit refers to the responsive interval of capacitance, preferably form 0.1 to 50 V; the charging time is 5-180 min, and the time of contact with the bacterial solution is more than one minute.

According to the sterilization method of the present disclosure, it is preferred to repeatedly charge to disinfection, further preferably two or more times.

The sterilization method according to the present disclosure, characterized in that by converting the mechanical energy to electrical energy via movement of human body, a repeated disinfection can be achieved by the cycles of charging and discharging.

According to the sterilization method of the present disclosure, the cyclic sterilization also inhibits the formation of biofilm.

In establishment of the present antibacterial system, the first step is preparing the supercapacitive material, full-charging the material with contact of circuit, post-charging material will interact with the bacteria, disturbing electron transfer of the respiratory chain of microorganism in short-term and inhibiting its growth and reproduction. With cycles of charging and discharging, the antimicrobial rate increases, the formation of the biofilm is suppressed, and no damage to its capacitive properties.

The beneficial effects of the present disclosure are:

The material with controllable supercapacitive properties is introduced into the surface of the material and charged. It can interact with bacteria after full charging and disturb respiratory chain of bacteria and inhibit its growth. The antibacterial system can quantitatively control the antibacterial process without affecting the biocompatibility of the material, and has the advantages of environmental protection and controllability.

In particular, the present disclosure has the following advantages over the conventionally designed antibacterial surface:

1. The supercapacitive properties of designed material in antimicrobial model, which prevents the antibiotic-resistance caused by the use of antibiotic.

2. Comparing the bioactive materials such as antibacterial peptides, this material prevents biosafety hazards.

3. According to the present disclosure, a titanium dioxide nanotube array can directly grow in situ on the surface of metal material, and using one-step annealing method to perform carbon deposition with; or zinc oxide nanorod in the present disclosure is prepared via hydrothermal method and reduced graphene oxide is prepared via electrodeposition method; modified surface is more firmly bonded to the material body without any leakage and loss of the contents.

4. The sterilization mechanism of antibacterial system is based on the electron transfer between modified surface and bacteria, which is more convenient and cleaner than the conventional bactericidal mechanism of drug release on surface.

5. The antibacterial system involved in the present disclosure is a local antibacterial system, which relies on the contact with bacteria, is more accurate to kill bacteria around the surgical site comparing with the ions or drugs release on surface, thereby achieving high-efficiency anti-infection.

6. This system can combine with a wearable energy device, with the movement of the body, converting mechanical energy to the electrical energy to make a cycle of charging and discharging that used in repeated disinfection.

7. The present disclosure of invention is more convenient, technology is mature and suitable for volume production.

8. The present disclosure of invention does not change biocompatibility with material, so it is safe used in vivo.

9. The present disclosure of invention can effectively inhibit the formation of bacterial biofilm on the surface of the material.

### BRIEF DESCRIPTION OF THE DRAWINGS

FIG. 1a shows the SEM (scanning electron microscope) image of titanium dioxide nanotube array (TNT-C-15) with the insets showing the corresponding enlarged and cross-sectional images (scale bar=500 nm), after vacuum annealing at heating rate of 15° C. min⁻¹ following anodization for 60 min under a scanning electron microscope; and, TNT represents an array of titanium dioxide nanotubes annealed in air.

FIG. 1b AFM (atomic force microscopy) image of shows the surface and cross section morphology of TNT-C-15 (anodization for 60 min and heating rate of vacuum annealing is 15° C. min⁻¹).

FIG. 1c shows the analysis of TNT-C-15 via STEM-EELS (scanning transmission electron microscope electron energy loss spectroscopy) maps (scale bar=50 mn)(anodization for 60 min and heating rate of vacuum annealing is 15° C. min⁻¹).

FIG. 1d shows the comparison of XRD patterns of TNT-C-15 and TNT (anodization for 60 min and heating rate of vacuum or air annealing is 15° C. min⁻¹).

FIG. 1e shows the high-resolution carbon electron spectra of samples from the surface (heating rate in air is 15° C. min⁻¹ (TNT), the heating rate of vacuum annealing is 5, 10, 15 and 20° C. min⁻¹, corresponding to TNT-C-5, TNT-C-10, TNT-C-15 and TNT-C-20, respectively).

FIG. 1f shows the high-resolution carbon X-ray photoelectron spectra of samples from the surface after sputtering for 6 min with Ar⁺ at a sputtering speed of 21 nm min⁻¹.

FIG. 2a shows the cyclic voltammetry (CV) curve of the samples annealed at different heating rates (heating rate in air is 15° C. min⁻¹, and 5, 10, 15 and 20° C. min⁻¹ in vacuum).

FIG. 2b shows the curve of the sample at galvanostatic charging-discharging (GCD) plots, annealed at different heating rates (heating rate in air is 15° C. min⁻¹, and 5, 10, 15 and 20° C. min⁻¹ in vacuum).

FIG. 3 shows the SEM image of hydrothermally synthesized zinc oxide nanorods magnetically sputtering with gold for 2 min.

FIG. 4a shows the CV curve of the capacitive properties of reduced graphene oxide prepared by electrodeposition in combination with hydrothermal method.

FIG. 4b shows the curves of capacitive properties—GCD of reduced graphene oxide prepared by electrodeposition in combination with hydrothermal method.

FIG. 5 shows a schematic diagram of charging a TNT-C sample.

FIG. 6a shows the post-charging antimicrobial rates of charged sample within 20 min (heating rate in air of 15° C. min⁻¹, and 5, 10, 15 and 20°C. min⁻¹ in vacuum); wherein P denotes direct current positive charging and N denotes direct current negative charging.

FIG. 6b shows the post-charging antimicrobial rates of charged sample with 180 min (heating rate in air of 15° C. min⁻¹, and 5, 10, 15 and 20° C. min⁻¹ in vacuum).

FIG. 7 shows the antibacterial rates of TNT-C against Staphylococcus epidermidis and Pseudomonas aeruginosa after charging for 20 min.

FIG. 8 shows the antibacterial rates of TNT-C-15 which is DC charging-discharging for three times (anodization for 60 min and heating rate of vacuum annealing is 15° C. min⁻¹).

FIG. 9 shows the 3D morphology of the fluorescent stained biofilm of TNT-C-15 after DC charging-discharging for 8 times (anodization for 60 min and heating rate of vacuum annealing is 15° C. min⁻¹); where DC represents direct current.

FIG. 10 shows the post-charging antimicrobial rates of TNT-C-15 against Escherichia coli and Staphylococcus aureus at AC (anodization for 60 min and heating rate of vacuum annealing is 15° C. min⁻¹).

FIG. 11 shows the post-charging antimicrobial rates of sample at 20 min, at different power supply and different charged times (the bacterium used is Escherichia coli); wherein AC represents alternating current; On 0.5 min, On 5 min and On 15 min represent charging for 0.5, 5 and 15 min, respectively.

FIG. 12 shows the post-charging antimicrobial rates of different ZnO samples against bacteria at 20 min, the time of gold sputtered on the surface of zinc oxide is 0, 2, 4, and 6 min (corresponding to ZnO, ZnO—Au-2, ZnO—Au-4, and ZnO—Au-6, respectively).

FIG. 13 shows the post-charging antimicrobial rates of the reduced graphene oxide-titanium alloy composite against bacteria at different time points.

### DETAILED DESCRIPTION

Pretreatment of Titanium Alloy and Supercapacitive Modification of Surface

The titanium alloy is cut to dimension of 30×30×0.5 mm, polished and ground, and ultrasonically cleaned in acetone, ethanol, and water in series for 10 min, and dried with nitrogen for future use.

The capacitive surface of the material can be designed by anodizing the surface of the titanium alloy to form titanium dioxide nanotube array of diameter of 10 nm to 500 nm; wherein the electrolyte used in anodization is ammonium fluoride (1-10%), methanol (1-10%), deionized water (1-10%) and ethylene glycol (70-95%); the voltage of anodization is 10-100 V; the reaction time is 20-1000 min; the sample is rinsed in 5 mL deionized water for 2 min and dried in nitrogen. The anodized nanotube array is placed into vacuum tube furnace annealing to acquire carbon concentrations (named as TNT-C) which enhances the capacitive properties, the temperature of anneal is 500-800° C., the time is 1-5 h, and the heating rate is 0.1-20° C. min⁻¹. The capacitance of the material can be quantitatively controlled by heating rate and temperature.

The capacitive surface of material can also be manufactured by hydrothermal method that growing zinc oxide nanorods doped with gold nanoparticles on the surface of titanium alloy. The specific operations are as follows: (1) Preparation of zinc oxide seed crystal: Weighing zinc acetate, sodium hydroxide and methanol and fully mixing to a 0.001-1 M solution at 50-70° C. for 1-10 h. The above solution is spin-coated on the treated titanium foil at a speed of 500-3000 r/min for 5-30 s to form a wet film, heating at 250 degrees for 5-20 min to volatilize the solvent and pyrolyzation the procedure is repeated 3-5 times, and cooling to acquire titanium foil with crystal seed layer. (2) Growth of zinc oxide nanorods: placing the sample from (1) in reactor of volume of 10-1000 mL, adding 0.001-1 M of 8-800 mL mixed solution of zinc nitrate and hexamethylenetetramine to reactor, sealing the reactor and heating in a muffle furnace at 90-120° C. for 8-48 h. The titanium foil growing with zinc oxide nanorods on the surface obtained from prior step will be ultrasonically cleaned. The gold particles with a particle size of 1-100 nm are sputtered by magnetron sputtering method to obtain the sample having supercapacitive characteristics.

In addition, the reduced graphene oxide is used as supercapacitive material to modify the titanium alloy. The pretreated titanium foil is sequentially immersed in 10%-30% nitric acid and 1-10 M sodium hydroxide solution for 5 min, washed with deionized water and dried at room temperature used as working electrode for electrodeposition. Contacting a 0.01-1 mg/mL electrodeposition solution of graphene oxide and aqueous ethanol solution (concentration of 10%-80%) with a reference electrode and a counter electrode to electrodeposition at 1-20 V direct current voltage at 40-50° C. for 1-60 min to acquire a graphene oxide layer. It was placed in 4% hydrazine solution and hydrothermally treated at 95° C. for 1 h to obtain a reduced graphene oxide-titanium alloy composite.

Charging and Sterilization Application of a Supercapacitive Material

Connecting the carbon-deposited titanium dioxide nanotube-modified titanium alloy above to an electrochemical workstation to test the capacitive property response voltage interval. Then the material is connected to DC or AC (peak-to-peak value of 2-40, frequency of 1 Hz-1 MHz) circuit, the voltage is set with reference to the responsive interval of capacitance (0.1-50 V), charging the capacitor for 5-180 min. Spreading the bacterial culture solution of 10-10⁶ CFU mL⁻¹ on the surface of post-charging material. After waiting for 1-180 min, the bacteria is cultured on agar plate and the physiological activity is measured, to analyze the antimicrobial effect.

### Embodiment 1

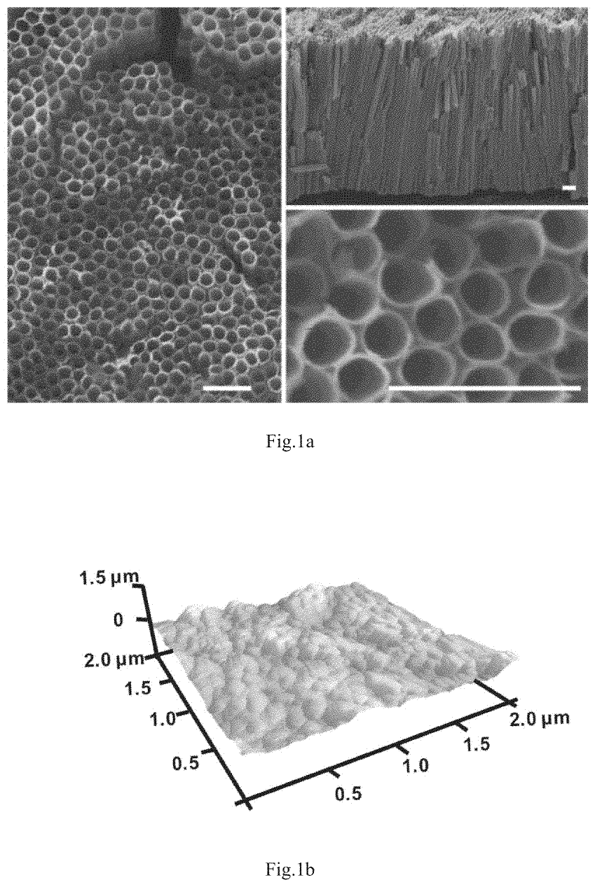

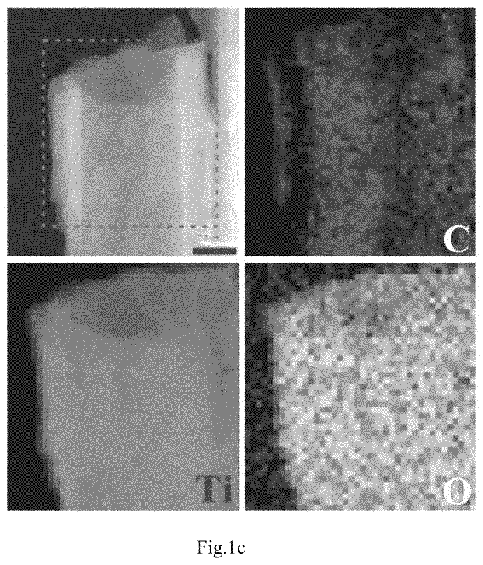

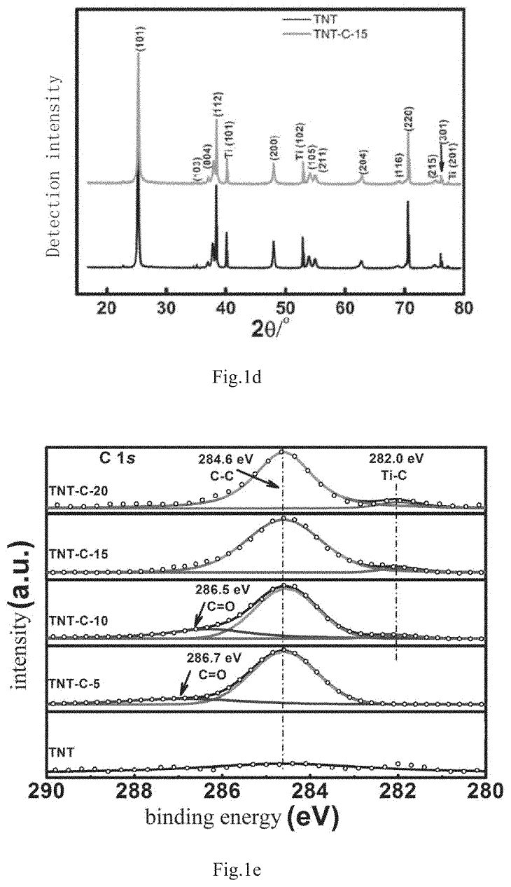

The titanium foil having a dimension of 30×30×0.5 mm is polished, grounded, and ultrasonically cleaned in acetone, alcohol, and deionized water in series. Connecting sample to positive electrode of the DC power supply to anodization, the electrolyte of anodization comprises ammonium fluoride (5.5%), methanol (5%), deionized water (5%) and ethylene glycol (70-90%), the voltage of anodization is 60 V, the time of anodization is 60 min, and the obtained sample is rinsed in 5 mL water for 2 min, dried in nitrogen. The anodized nanotube array is placed in a vacuum tube furnace annealing to obtain deposited carbon, which improves the rate of electron transfer of semiconductor titanium dioxide and reduces the rate of neutralization of positive and negative charges, increasing the specific surface area to enhance the capacitive properties, the temperature of anneal is 500° C., the time is 3 h, and the heating rate is 15° C. min⁻¹, the sample annealed in air under the same conditions is conducted as control group with no deposited carbon. The SEM image of the microscopic morphology of sample surface is shown in FIG. 1a. As shown in FIG. 1a, the anodized titanium dioxide nanotubes has an outer diameter of 160 nm, wall thickness of 25 nm, and nanotubes length of 10 μm. Similar results are shown in AFM image (FIG. 1b). Comparing with titanium dioxide nanotubes annealed in air, the titanium dioxide nanotubes annealed in argon do not cause a significant change in morphology. It can be concluded that deposited carbon did not cause a significant change in the microscopic morphology to titanium dioxide nanotube array.

### Embodiment 2

The surface of the sample obtained from Embodiment 1 is tested by elemental content analysis. The STEM-EELS maps indicate carbon was evenly precipited on the wall of titanium dioxide nanotube (shown in FIG. 1c). The main peak of anatase crystalline titanium dioxide (2θ=25.3° (101), 48.0° (200), and 70.3° (220)) is shown in XRD patterns (FIG. 1d). Further researches of X-ray photoelectron spectra (XPS) revealed that the distribution pattern of carbon elements on the surface of the sample is dominated by C-C bond (FIG. 1e), but the C—Ti bond turns to the dominant way after sputtering for 6 min (FIG. 1f), which can be explained by that the carbon partially substitutes oxygen in titanium dioxide and precipitating evenly. The elemental analysis results above indicate the titanium dioxide nanotube array with uniformly distributed carbon was prepared.

### Embodiment 3

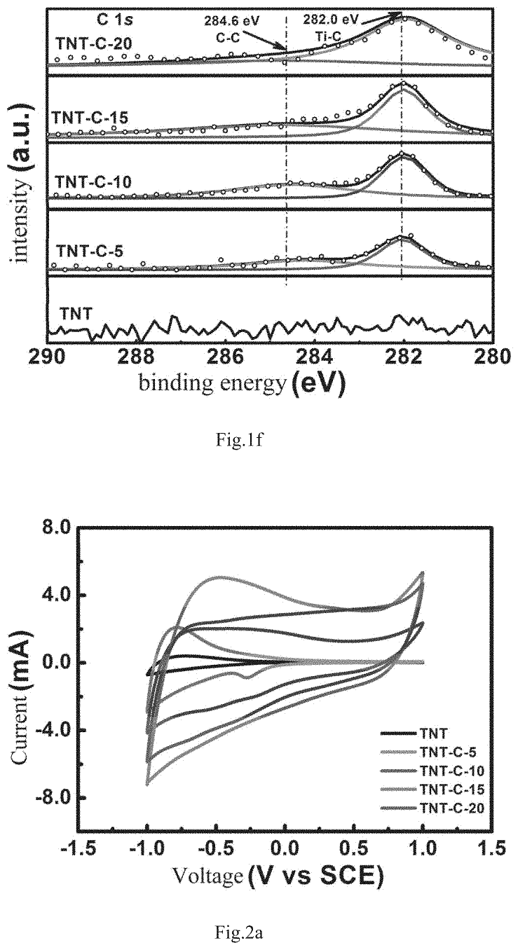

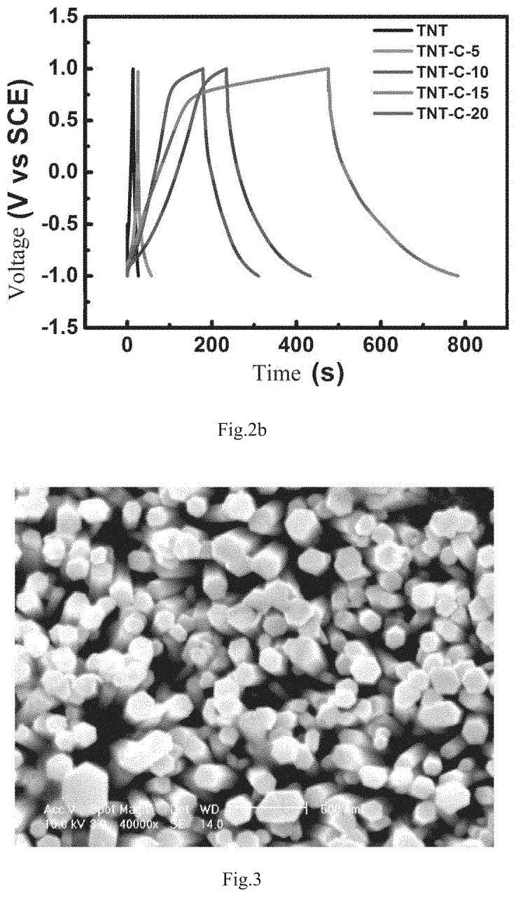

Capacitance analysis of the prepared sample is carried out by electrochemical workstation. It can be detected obviously under 15° C. min⁻¹ annealing condition that the sample has electric double layer capacitive properties(FIG. 2a), and the sample can accumulate more charges under 15° C. min⁻¹ annealing condition (FIG. 2b), indicating that there can have more electron transfer when antibacterial was carried out thereafter.

### Embodiment 4

Titanium foil having a dimension of 30×30×0.5 mm is polished, grounded, and ultrasonically cleaned in acetone, alcohol, and deionized water in series. 0.219 g of zinc acetate, 0.12 g of sodium hydroxide and 100 mL of methanol are weighed to formulate a mixed solution, which is stirred at 60° C. for 2 h to mix well. The mixed solution is spin-coated on the treated titanium foil at a speed of 3000 r/min for 20 s to obtain a wet film, heating it at 250 degrees for 5 min to volatilize the solvent and pyrolyzation, this procedure repeats 3 times, cooling to acquire titanium foil with a crystal seed layer. Placing sample into a reactor of 20 mL volume, prepare 10 mL of mixed solution of zinc nitrate and hexamethylenetetramine with a concentration of 100 μM, add the mixed solution to the reactor. Sealing the reactor and place it into a muffle furnace at 90° C. for 10 h. The sample is taken out and the obtained titanium foil growing with zinc oxide nanorods is ultrasonically cleaned for 10 s. The gold particles are sputtered on surface of sample by magnetron sputtering method for 2 min to acquire a capacitive characteristic. the SEM image of sample is shown in FIG. 3.

### Embodiment 5

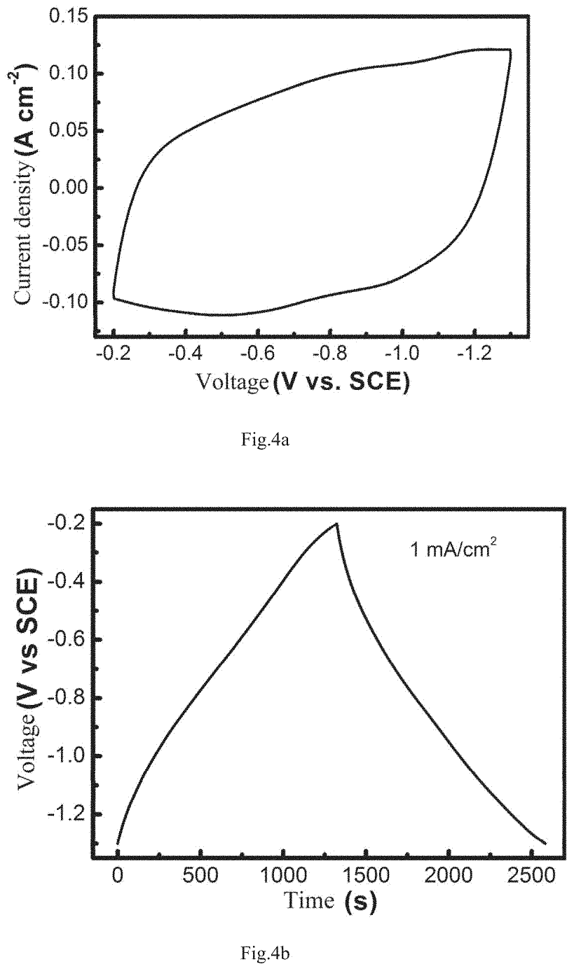

The ground and cleaned titanium foil is sequentially immersed in 20% nitric acid and 5 M sodium hydroxide solution for 5 min, washed in deionized water and dried at room temperature used as a working electrode to electrodeposition. Adding graphene oxide to 30% aqueous ethanol solution to prepare a concentration of 0.3 mg/mL of electrodeposition solution, connecting with a reference electrode and a counter electrode to electrodeposition at direct current voltage of 10 V at 40° C. for 20 min to obtain a graphene oxide layer. Putting it into 4% hydrazine solution and hydrothermally treating at 95° C. for 1 h to obtain a reduced graphene oxide-titanium alloy composite which are connected to electrochemical workstation to analyze its capacitive characteristics, the figures of CV curve and curve of GCD are shown in FIGS. 4a and 4b.

### Embodiment 6

The sample obtained in Embodiment 1 is subjected to DC charging, the charging voltage is 2 V, and the charging time is 20 min. The schematic diagram of charging is shown in FIG. 5.

### Embodiment 7

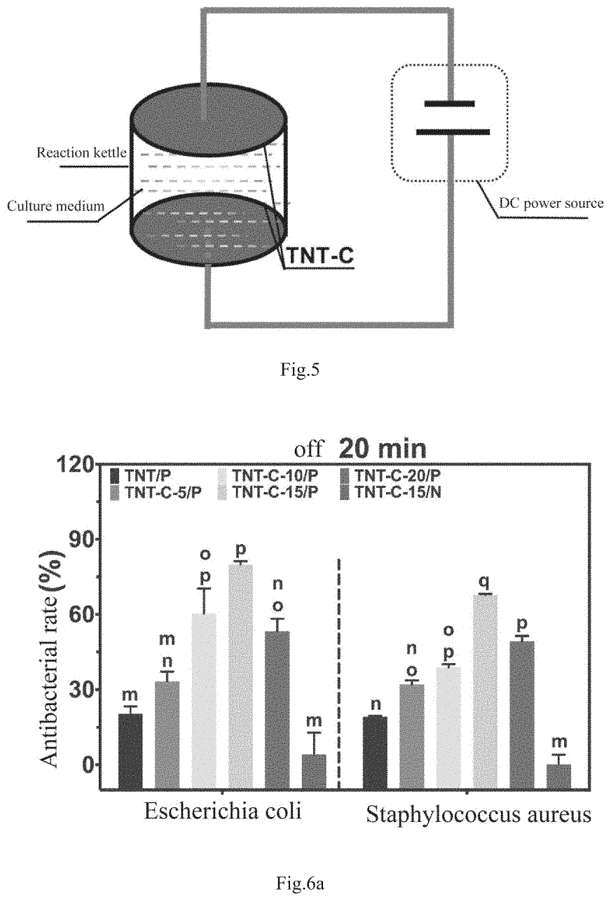

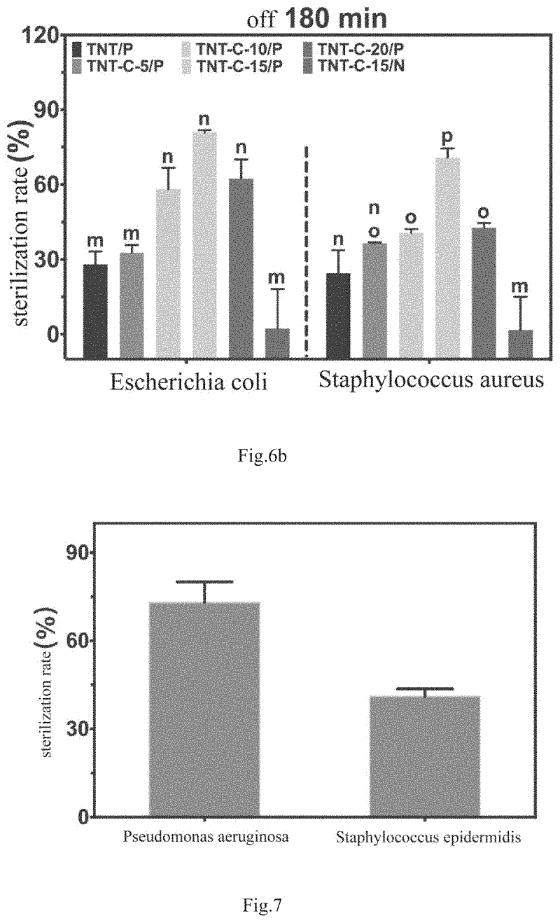

The fully charged sample in Embodiment 6 is taken out and applied to the antibacterial (Staphylococcus aureus and Escherichia coli) test. The antimicrobial effect is evaluated by the coated plate counting method and the results are shown in FIG. 6. For samples with larger capacitance, Higher sterilization rate can be achieved after fully charged. For example, about 80% and about 70% of sterilization rates to Escherichia coli and Staphylococcus aureus can be achieved at 15° C. min⁻¹ when interacted with bacteria for 20 minutes after fully charged (FIG. 6a). Extending the interaction time of the material to the bacteria for up to 180 min did not significantly increase the antibacterial effect (FIG. 6b), indicating that the antibacterial process occurred at early stage of the contact. In addition, the sterilizing efficiency of the positively charged surface of the sample is significantly higher than that of the negatively charged ones.

### Embodiment 8

The antibacterial operation in Embodiment 7 is applied to two other bacteria (Pseudomonas aeruginosa and Staphylococcus epidermidis) to further verify its antibacterial effect, and the results showed that TNT-C-15 could realize about 75% and about 45% of antibacterial effects against Pseudomonas aeruginosa and Staphylococcus epidermidis within 20 min after being positively charged (FIG. 7). Compared with the antibacterial results in Embodiment 7, it can be seen that the antibacterial system based on the supercapacitor material has a significantly higher antibacterial effect against Gram-negative bacteria than Gram-positive bacteria.

### Embodiment 9

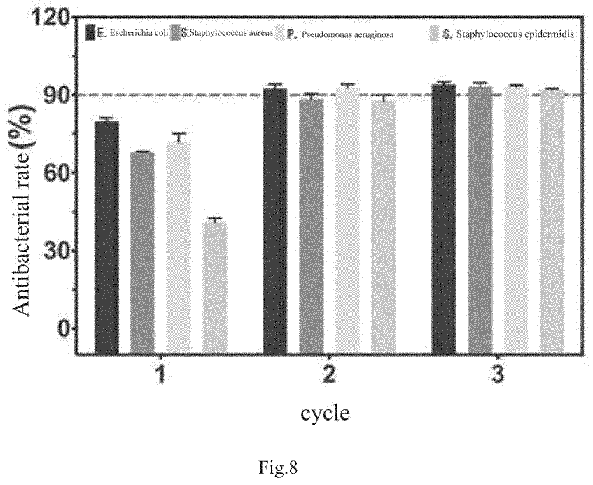

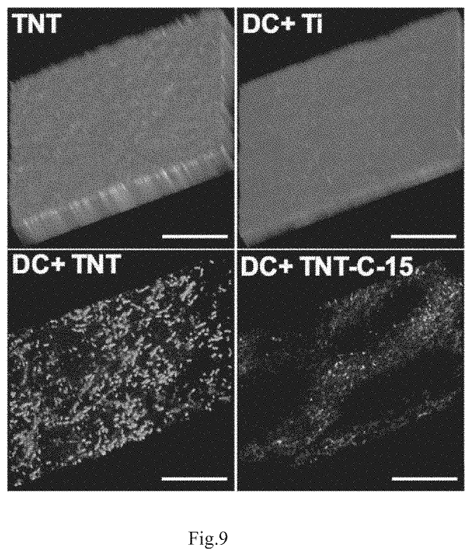

In order to improve the antibacterial efficiency, the bacteria are collected after sterilizing for 20 minutes in Embodiment 7, and the sample is recharged (positively charged), and then the collected bacteria are added to the surface of the material for secondary sterilization, and the antibacterial results are shown in FIG. 8. The results showed that the sterilization rate of the four bacteria can be increased to about 90% in the second cycle charging process, and the antibacterial rate of greater than 90% can be achieved after three cycles of charging.

### Embodiment 10

The bacteria on the material after sterilization for 20 min in Embodiment 7 are cultured in a bacterial culture medium at 37° C., the material is charged every 6 h, and co-cultured to 48 h. The formation of biofilm is observed by fluorescent staining method, as shown in FIG. 9. A strong biofilm is formed on the uncharged titanium dioxide nanotubes. The biofilm is also formed on the DC-charged titanium sheets but the thickness is significantly lower than that of the uncharged titanium dioxide group. Died bacteria could be obviously detected on the charged and discharged titanium dioxide and carbon deposited titanium dioxide and no continuous biofilm is formed thereon, these results proved that the titanium alloy based on supercapacitor material can effectively inhibit the formation of biofilm during charge and discharge, and the suppression effect is positively correlated with the capacitance.

It is confirmed by experiments that the titanium dioxide nanotube array with diameter of 160 nm is prepared by Redox method , which is annealed in argon (annealing temperature of 500° C., annealing time of 3 h, and heating rate of 15° C. min⁻¹) to obtain the carbon deposited titanium dioxide nanotube array, which has supercapacitor characteristics. It is charged with a DC power source (2 V) for 15 min, and a sterilization rate of more than 80% can be achieved within 20 min, and more than 90% of sterilization rate can be achieved after three cycles of charging and formation of biofilm can be effectively inhibited. in the body, after the bacteria breed, it is often easy to form a biofilm with extracellular polymer matrix, specific structure and stronger resistance, which can cause serious postoperative infection, the anti-biofilm efficacy of the present disclosure can significantly reduce the risk of postoperative infection.

### Embodiment 11

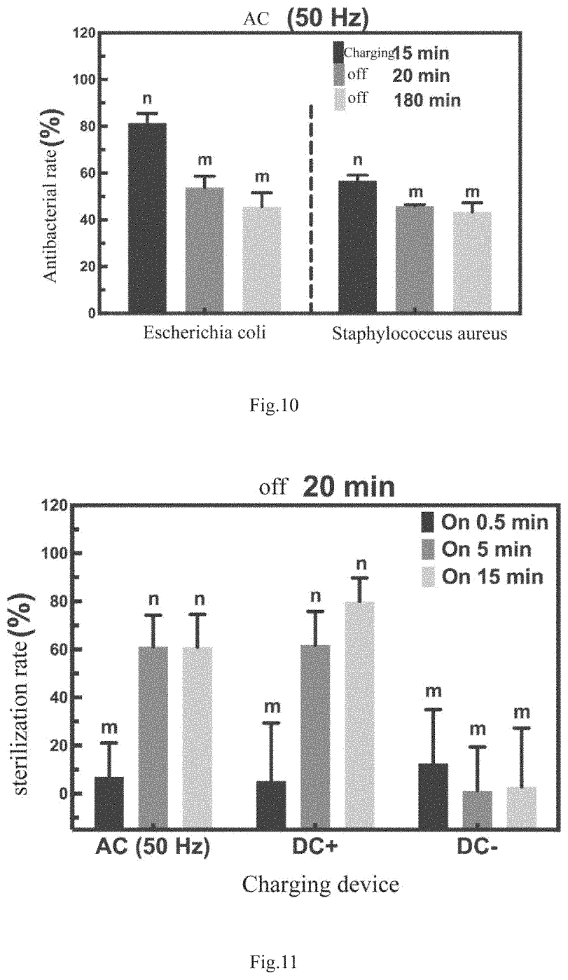

The TNT-C-15 sample in Embodiment 1 is subjected to AC charging with a voltage peak-to-peak value of 2 V, a frequency of 50 Hz, and a charging time of 15 min. The fully charged sample is taken out and applied to the antibacterial (Staphylococcus aureus and Escherichia coli) test. The antibacterial effect is evaluated by the coated plate counting method, and the results are shown in FIG. 10. The results showed that about 80% and 60% of antibacterial rate against E. coli and S. aureus can be achieved during the 15 min charging period. After de-energized, more than 40% of antibacterial rate could still be achieved within 20 min and 180 min when contacting with the bacteria. It is indicated that the alternating current can charge the material in the present disclosure to achieve a sterilization effect by using its capacitance.

### Embodiment 12

The sample in Embodiment 1 is charged (the AC-DC parameters are the same as above) for different periods of time to obtain samples carrying different charge densities, and then the sample is contacted with E. coli (its concentration is the same as above), and the sterilization effect within 20 min is judged by the coated plate counting method. The results are as shown in FIG. 11. For samples treated with AC and DC positively, the sample can achieve a higher sterilization rate within 20 min as the charging time is extended. This result indicated that the longer the charging time of the material with capacitive properties, the more charges are accumulated on the surface, and the higher is the sterilization efficiency

### Embodiment 13

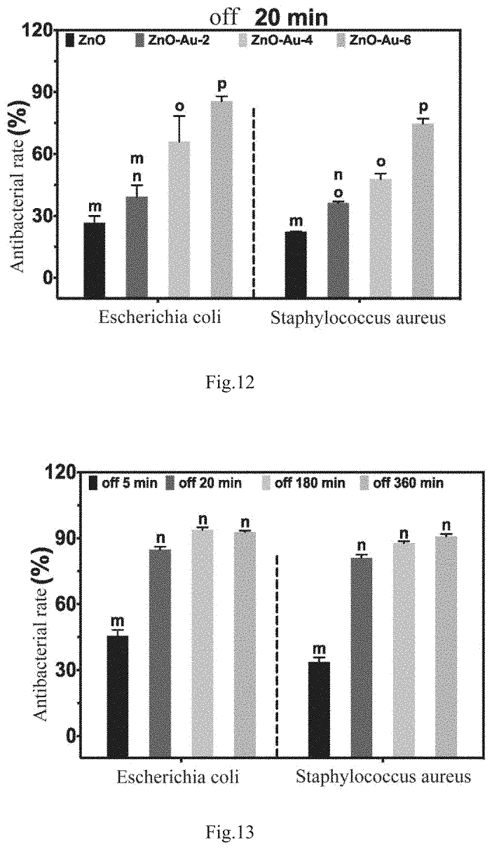

The ZnO sample obtained in Embodiment 4 is subjected to DC charging, the charging voltage is 2 V, and the charging time is 20 min. The schematic diagram of charging is shown in FIG. 5 (the TNT-C sample is replaced with a ZnO sample). The fully charged sample is taken out and applied to the antibacterial (Staphylococcus aureus and Escherichia coli) test. The antibacterial effect is evaluated by the coated plate counting method, and the results are shown in FIG. 12. For samples with larger capacitance, higher sterilization rate can be achieved after fully charged. For example, ZnO—Au-6, about 90% and about 80% of sterilization rates to Escherichia coli and Staphylococcus aureus can be achieved when interacted with bacteria for 20 minutes after fully charged (FIG. 12).

### Embodiment 14

The sample obtained in Embodiment 5 is subjected to DC charging, the charging voltage is 1.5 V, and the charging time is 20 min. The schematic diagram of charging is shown in FIG. 5 (the TNT-C sample is replaced with a reduced graphene oxide-titanium alloy composite sample). The fully charged sample is taken out and applied to the antibacterial (Staphylococcus aureus and Escherichia coli) test. The antibacterial effect is evaluated by the coated plate counting method. The results are shown in FIG. 13. The bactericidal effect is gradually increased during the first 20 min of the interaction of the sample with the bacteria after charging, and the bactericidal effect is slowly increased within the treatment time of 20-360 min, and finally more than 90% of the sterilization rate can be achieved.

1. A material with supercapacitance modified surface, comprising:
a material body; and
a supercapacitance layer on surface;
wherein the material body is selected from a metal material or other conductors, the supercapacitance layer refers to a functional layer having a surface capacitance greater than 10 mF·cm⁻².

2. The material according to claim 1, wherein the metal material is selected from the group consisting of titanium or an alloy thereof, aluminum or an alloy thereof, stainless steel, nickel or an alloy thereof, manganese or an alloy thereof, tungsten or an alloy thereof, zinc or an alloy thereof; the other conductors are selected from the group consisting of conductive polymers, including polypyrrole, polyacetylene, polythiophene, polyaniline; the surface capacitance of the supercapacitance layer is greater than 50 mF·cm⁻², preferably greater than 100 mF·cm⁻².

3. The material according to claim 2, wherein the metal material is selected from the group consisting of a titanium alloy, an aluminum alloy, a stainless steel, a nickel alloy, and a zinc alloy; and the supercapacitance layer is selected from the group consisting of a titanium dioxide nanotube array layer, a zinc oxide nanorod layer, or a reduced graphene oxide.

4. The material according to claim 3, wherein the titanium dioxide nanotubes or zinc oxide nanorods have a diameter of 10 nm to 1000 nm, preferably 20 to 800 nm, most preferably 50 to 500 nm; and a pipe diameter of 500 nm to 10 μm.

5. The material according to claim 4, wherein the titanium dioxide nanotube array layer further comprises deposited carbon; and the zinc oxide nanorod layer is doped with silver, gold, copper or platinum nanoparticles.

6. A preparation method of material with supercapacitance modified surface according to claim 1, comprising: anodizing the surface of metal material, the electrolyte used to anodize is a mixed solution of an ammonium salt, a lower alcohol, water, and a polyol.

7. The preparation method according to claim 6, wherein the ammonium salt is selected from an ammonium halide, preferably ammonium fluoride; the lower alcohol is selected from methanol or ethanol; and the polyol is selected from ethylene glycol; the voltage of anodization is 10-100 V and the time of anodization is 20-1000 min.

8. The preparation method according to claim 6, wherein the array of nanotubes obtained from anodization is placed in a vacuum tube furnace annealing in vacuum to achieve carbon deposition, so as to enhance the capacitive characteristics; and the temperature of vacuum anneal is 500-800° C., the annealing time is 1 to 5 h, and the heating rate is 1 to 20° C. min⁻¹.

9. A preparation method of material with supercapacitance modified surface according to claim 1, comprising:
growing zinc oxide nanorods on the surface of the metal material by a hydrothermal method and sputtering the doped silver, gold, copper or platinum nanoparticles by magnetron sputtering, the specific steps are as follows:
(1) preparation of zinc oxide seed crystal: dissolving zinc acetate and a strong base in a lower alcohol, quickly spin-coating on the surface of the metal material to obtain a wet film, heating to volatilize the solvent and pyrolyzation to obtain a metal material with a crystal seed layer;
(2) growth of zinc oxide nanorods: placing the sample from (1) into a reactor, adding a mixed aqueous solution of a zinc salt and a base, sealing and heating the reactor; sputtering silver, gold, copper or platinum nanoparticles by magnetron sputtering; the base is preferably hexamethylenetetramine, sodium hydroxide, potassium hydroxide, calcium hydroxide, and aqueous ammonia.

10. A preparation method of the material with supercapacitance modified surface according to claim 1, comprising: using metal material as working electrode to electrodeposition;
adding graphene oxide into alcoholic aqueous solution as eletro-deposited solution and connecting it with a reference electrode and a counter electrode, electrodeposition with DC to obtain a layer of graphene oxide; hydrothermal treatment of the obtained sample in hydrazine solution to obtain a reduced graphene oxide-metal composite.

11. A sterilization method, comprising using the material with supercapacitance modified surface according to claim 1.

12. The sterilization method according to claim 11, further comprising: charging the material to DC or an AC circuit and interacting with the bacterial cultural solution; the electric charge of charging process is preferably positive charge.

13. The sterilization method according to claim 12, wherein the voltage of the circuit is set referring to the response interval of capacitance, the charging time is 5-180 min, and the time of interaction with the bacterial cultural solution is more than one minute.

14. The sterilization method according to claim 11, wherein the charging sterilization process is carried out for several times, preferably two or more times.

15. The sterilization method according to claim 14, wherein the cyclical sterilization is achieved by converting the mechanical energy from body movement to the electrical energy repeatedly to charging-discharging material.

16. A sterilization method, comprising using the material with supercapacitance modified surface according to claim 2.

17. A sterilization method, comprising using the material with supercapacitance modified surface according to claim 3.

18. A sterilization method, comprising using the material with supercapacitance modified surface according to claim 4.

19. The sterilization method according to claim 12, wherein the charging sterilization process is carried out for several times, preferably two or more times.

20. The sterilization method according to claim 13, wherein the charging sterilization process is carried out for several times, preferably two or more times.
